(19) United States
(12) Patent Application Publication
(10) Pub. No.: US 2021/0071219 A1
(43) Pub. Date: Mar. 11, 2021

(54) Simplified Process For Producing Maltodextrin and Specialty Syrups

(71) Applicant: DANISCO US INC, Palo Alto, CA (US)

(72) Inventors: Vipul GOHEL, Palo Alto, CA (US); Kannan RANGANATHAN, Palo Alto, CA (US); Gang DUAN, Palo Alto, CA (US)

(21) Appl. No.: 17/053,006

(22) PCT Filed: Apr. 30, 2019
(86) PCT No.: PCT/US2019/029843

§ 371 Date: Nov. 4, 2020

(30) Foreign Application Priority Data
May 4, 2018 (IN) 201811016966

(51) Int. Cl. C12P 19/14; C12P 19/08; C12N 9/99

(52) U.S. Cl. CPC C12P 19/14; C12P 19/08; C12N 9/99; C12Y 302/01003; C12Y 302/01002; C12Y 302/01133; C12Y 302/01041; C12Y 302/01001

(57) ABSTRACT

Disclosed are compositions and methods relating to a simplified process for producing maltodextrin and specialty syrups using fewer enzymes and less complicated conditions than required for contemporary enzymatic processes.

### FIELD OF THE INVENTION

The present compositions and methods relate to a simplified process for producing maltodextrin and specialty syrups using fewer enzymes and less complicated conditions than are required for current enzymatic processes.

### BACKGROUND

Starch based sweeteners such as corn syrup, glucose syrups, maltodextrins and high fructose syrups are conventionally produced by liquefying starch using acid or enzyme treatment followed by enzymatic saccharification until a desired DE is achieved. The physical properties of corn syrups vary significantly depending on their composition. Corn syrup is classified into four types based on dextrose equivalents (DE). Type 1 corn syrup has a DE between 20 and 38. Type 2 corn syrup has a DE between 38-58. Type 3 corn syrup has a DE between 58-73. Type 4 corn syrup has a DE above 73. The Table in FIG. 1 depicts in greater detail the DE of various syrups being produced conventional processes.

Enzymatic processing has become favored over the acid-treatment process and specialty syrups with DE ranging from 34-43 are currently being produced by a combination of liquefaction and partial saccharification assisted by α-amylase and maltogenic enzymes such as maltogenic amylase, D-amylase, pullulanase, and glucoamylase. These maltogenic enzymes are used either in combination or individually depending on the sugar profile desired.

In addition to a battery of enzymes, the conversion of dextrinized starch post-liquefaction, calls for a series of steps that require 16-18 hours and need to be followed rigorously. These steps include (i) reduction of the pH to less than 4.50 at 90° C. using HCl to inactivate the liquifying α-amylase (preferably pH 4.20-4.30), (ii) cooling the liquefact to 60° C. for optimal performance of glucoamylase or other maltogenic enzyme, (iii) heating the saccharified liquifact to 85-90° C. to inactivate the glucoamylase or other maltogenic enzyme and (iv) cooling the saccharified liquifact to 60° C. to concentrate the product to a desired level of DS. This process is cumbersome and energy, time and manpower-intensive.

The need exists for improved processes for producing maltodextrin powder and specialty syrups.

### SUMMARY

The present compositions and methods relate to a simplified process for producing maltodextrin and specialty syrups. Aspects and embodiments of the present compositions and methods are summarized in the following separately-numbered paragraphs:

1. In one aspect, a method for producing a maltodextrin and/or a specialty syrup is provided, comprising contacting a starch substrate with an α-amylase (EC 3.2.1.1) capable of producing, in the substantial absence of a maltogenic enzyme selected from the group consisting of maltogenic amylase (EC 3.2.1.133), β-amylase (EC 3.2.1.2), pullulanase (EC 3.2.1.41), glucoamylase (EC 3.2.1.3) and combinations, thereof, a syrup comprising a DE profile equivalent to the DE profile produced by conventional, multi-enzyme, acid pretreatment conditions that includes a maltogenic enzyme, wherein the method substantially obviates at least one pH adjustment or temperature adjustment step in an otherwise identical process utilizing a different, conventional liquifying α-amylase.

2. In some embodiments, the method of paragraph 1 is performed in the absence of a maltogenic enzyme, with the exception of the α-amylase, which may have maltogentic amylase activity.

3. In some embodiments, the method of paragraph 2 is performed in the absence of any maltogenic enzyme, with the exception of the α-amylase, which may have maltogentic amylase activity.

4. In some embodiments of the method of any of paragraphs 1-3, the process step is selected from the group consisting of reducing the pH of a liquefact to inactivate a different, conventional liquifying α-amylase, cooling the liquefact to promote optimal performance of a maltogenic enzyme, heating a saccharified liquifact to inactivate the maltogenic enzyme, and cooling the saccharified liquifact to concentrate the product.

5. In some embodiments of the method of any of paragraphs 1-4, the α-amylase is from a Cytophaga sp.

6. In some embodiments of method of any of paragraphs 1-5, the α-amylase is the α-amylase from Cytophaga sp. having the amino acid sequence of SEQ ID NO: 1, or a variant, thereof.

7. In some embodiments of method of any of paragraphs 1-5, the conventional liquifying α-amylase is from Bacillus.

These and other aspects and embodiments of the compositions and methods will be apparent from the present description and drawings.

### BRIEF DESCRIPTION OF THE DRAWINGS

FIG. 1 shows a Table detailing the DE of various syrups being produced by current enzymatic processes.

### DETAILED DESCRIPTION

### 1. Introduction

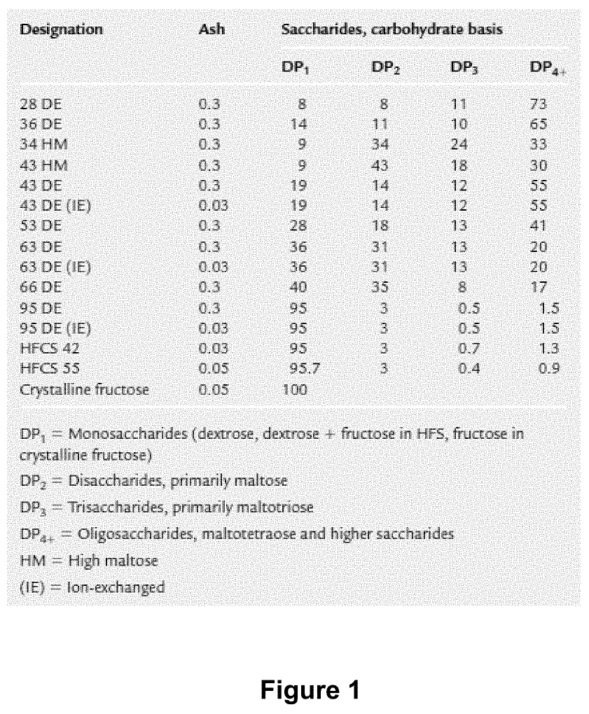

Described are compositions and methods relating to a simplified process for producing maltodextrin and specialty syrups using fewer enzymes and less complicated conditions than are required for current enzymatic processes. It has been discovered that certain α-amylases have the ability to produce maltodextrin and specialty syrups of Types 1 and 2 (FIG. 1) with DE ranging from 30-46, which match the profile of commercial syrups produced using a more traditional, acid-enzyme process (see, e.g., Shukla, P. and Pletschke, B. I. (eds.) Advances in Enzyme Biotechnology, Springer Science & Business Media, 2013). The improved process does not require additional maltogenic enzymes and requires much simpler process conditions. The benefits of the present compositions and methods include (i) energy savings, as the result of fewer cooling and heating steps, (ii) increased plant throughput and smoother operations and (iii) time saving resulting from the elimination of cooling and heating steps.

Prior to describing the various aspects and embodiments of the present compositions and methods, the following definitions and abbreviations are described.

### 2. Definitions and Abbreviations

In accordance with this detailed description, the following abbreviations and definitions apply. Note that the singular forms “a.” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art. The following terms are defined, below, for clarity.

### 2.1. Abbreviations and Acronyms

The following abbreviations/acronyms have the following meanings unless otherwise specified:

EC Enzyme Commission

DE Dextrose equivalents

DP Degree of polymerization

GA glucoamylase

ppm parts per million, e.g., μg protein per gram dry solid

sp. species

w/v weight/volume

w/w weight/weight

v/v volume/volume

wt % weight percent

° C. degrees Centigrade

dH₂O or DI deionized water

dIH₂O deionized water, Milli-Q filtration

g or gm grams

μg micrograms

mg milligrams

kg kilograms

μL and μl microliters

mL and ml milliliters

mm millimeters

μm micrometer

M molar

mM millimolar

μM micromolar

U units

min(s) minute/minutes

hr(s) hour/hours

N normal

T metric tonnes

### 2.2. Definitions

As used herein, the term “starch” refers to any material comprised of the complex polysaccharide carbohydrates of plants, comprised of amylose and amylopectin with the formula (C₆H₁₀O₅)ₓ, wherein X can be any number. The term includes plant-based materials such as grains, cereal, grasses, tubers and roots, and more specifically materials obtained from wheat, barley, corn, rye, rice, sorghum, brans, cassava, millet, milo, potato, sweet potato, and tapioca. The term “starch” includes granular starch. The term “granular starch” refers to raw, i.e., uncooked starch, e.g., starch that has not been subject to gelatinization.

As used herein, an “α-amylase” (EC 3.2.1.1) is an enzyme that catalyses endohydrolysis of (1->4)-α-D-glucosidic linkages in polysaccharides containing three or more (1->4)-α-linked D-glucose units.

As used herein, a “β-amylase” (EC 3.2.1.2) is an enzyme that catalyses hydrolysis of (1->4)-α-D-glucosidic linkages in polysaccharides so as to remove successive maltose units from the non-reducing ends of the chains.

As used herein, a “pullulanase” (EC 3.2.1.41) is an enzyme that catalyses hydrolysis of (1->6)-α-D-glucosidic linkages in pullulan, amylopectin and glycogen, and in the α- and α-limit dextrins of amylopectin and glycogen.

As used herein, a “glucoamylase” (EC 3.2.1.3) is an enzyme that catalyses hydrolysis of terminal (1->4)-linked α-D-glucose residues successively from non-reducing ends of the chains with release of β-D-glucose.

As used herein, a “maltogenic amylase” (EC 3.2.1.133) is an enzyme that catalyses hydrolysis of (1->4)-α-D-glucosidic linkages in polysaccharides so as to remove successive α-maltose residues from the non-reducing ends of the chains.

As used herein, the term “liquefaction” or “liquefy” means a process by which starch is converted to less viscous and shorter chain dextrins.

As used herein, the terms, “wild-type,” “parental,” or “reference,” with respect to a polypeptide, refer to a naturally-occurring polypeptide that does not include a man-made substitution, insertion, or deletion at one or more amino acid positions.

As used herein, the term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

As used herein, “combinatorial variants” are variants comprising two or more mutations, e.g., 2, 3, 4, 5, 6, 7, 8, 9, 10, or more, substitutions, deletions, and/or insertions.

As used herein, the term “recombinant” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding an amylase is a recombinant vector.

As used herein, the terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof, includes, but is not limited to, a culture broth containing secreted polypeptide expressed in a heterologous host cell.

As used herein, the terms “thermostable” and “thermostability,” with reference to an enzyme, refer to the ability of the enzyme to retain activity after exposure to an elevated temperature. The thermostability of an enzyme, such as an amylase enzyme, is measured by its half-life (t/2) given in minutes, hours, or days, during which half the enzyme activity is lost under defined conditions. The half-life may be calculated by measuring residual α-amylase activity following exposure to (i.e., challenge by) an elevated temperature.

As used herein, a “pH range,” with reference to an enzyme, refers to the range of pH values under which the enzyme exhibits catalytic activity.

As used herein, the terms “pH stable” and “pH stability.” with reference to an enzyme, relate to the ability of the enzyme to retain activity over a wide range of pH values for a predetermined period of time (e.g., 15 min., 30 min., 1 hour).

As used herein, the term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

As used herein, the term “nucleic acid” encompasses DNA, RNA, heteroduplexes, and synthetic molecules capable of encoding a polypeptide.

As used herein, “hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M sodium citrate, pH 7.0). Hybridized, duplex nucleic acids are characterized by a melting temperature (Tm), where one half of the hybridized nucleic acids are unpaired with the complementary strand. Mismatched nucleotides within the duplex lower the Tm. A nucleic acid encoding a variant α-amylase may have a Tm reduced by 1° C.-3° C. or more compared to a duplex formed between the nucleotide of SEQ ID NO: 2 and its identical complement.

As used herein, “biologically active” refer to a sequence having a specified biological activity, such an enzymatic activity.

As used herein, the term “specific activity” refers to the number of moles of substrate that can be converted to product by an enzyme or enzyme preparation per unit time under specific conditions. Specific activity is generally expressed as units (U)/mg of protein.

As used herein, “water hardness” is a measure of the minerals (e.g., calcium and magnesium) present in water.

As used herein, “percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

Deletions are counted as non-identical residues, compared to a reference sequence.

As used herein, the term “dry solids content” (ds or DS) refers to the total solids of a slurry in a dry weight percent basis.

As used herein, the term “slurry” refers to an aqueous mixture containing insoluble solids.

As used herein, the term “about” refers to 15% to the referenced value.

### 3. Suitable α-Amylases

An aspect of the present compositions and methods are α-amylase enzymes that can used in the substantial or complete absence of additional enzymes having maltogenic amylase activity. An exemplary α-amylase is the wild-type α-amylase from a Cytophaga sp. (herein referred to as “CspAmy2 amylase”). which was previously described by Jeang, C-L et al. ((2002) Applied and Environmental Microbiology, 68:3651-54). The amino acid sequence of the mature form of the CspAmy2 α-amylase polypeptide is shown, below, as SEQ ID NO: 1:

AATNGTMMQY FEWYVPNDGQ QWNRLRTDAP YLSSVGITAV
WTPPAYKGTS QADVGYGPYD LYDLGEFNQK GTVRTKYGTK
GELKSAVNTL HSNGIQVYGD VVMNHKAGAD YTENVTAVEV
NPSNRNQETS GEYNIQAWTG FNFPGRGTTY SNFKWQWFHF
DGTDWDQSRS LSRIFKFRGT GKAWDWEVSS ENGNYDYLMY
ADIDYDHPDV VNEMKKWGVW YANEVGLDGY RLDAVKHIKF
SFLKDWVDNA RAATGKEMET VGEYWQNDLG ALNNYLAKVN
YNQSLFDAPL HYNFYAASTG GGYYDMRNIL NNTLVASNPT
KAVTLVENHD TQPGQSLEST VQPWFKPLAY AFILTRSGGY
PSVFYGDMYG TKGTTTREIP ALKSKIEPLL KARKDYAYGT
QRDYIDNPDV IGWTREGDST KAKSGLATVI TDGPGGSKRM
YVGTSNAGEI WYDLIGNRTD KITIGSDGYA TFPVNGGSVS
VWVQQ

CspAmy2 α-amylase proved to by an extremely versatile molecule that was suitable for both grain processing applications, which require low pH activity and thermostability, and cleaning applications, which require medium to high pH activity and surfactant stability.

Variants of CspAmy2 α-amylase have been made that have improved properties in one or the other application, however, such enzymes remain versatile despite being tailored for a given application.

In SEQ ID NO: 1, above, R178, G179, T180 and G181, are underlined. In some embodiments, the variant α-amylases further include a deletion in this XtG/StX2G2 motif, which is adjacent to the calcium-binding loop. In some embodiments, the variant α-amylases include adjacent, pair-wise deletions of amino acid residues corresponding to R178 and G179, or T180 and G181. A variant of the Cytophaga sp. α-amylase having a deletion of both R178 and G179 (herein, “CspAmy2-v1”) has also been described (Shiau, R-J. et al. (2003) Applied and Environmental Microbiology, 69:2383-85). The amino acid sequence of the mature CspAmy2-v1 a-amylase polypeptide is shown below as SEQ ID NO: 2:

AATNGTMMQY FEWYVPNDGQ QWNRLRTDAP YLSSVGITAV
WTPPAYKGTS QADVGYGPYD LYDLGEFNQK GTVRTKYGTK
GELKSAVNTL HSNGIQVYGD VVMNHKAGAD YTENVTAVEV
NPSNRNQETS GEYNIQAWTG FNFPGRGTTY SNFKWQWFHF
DGTDWDQSRS LSRIFKFTGK AWDWEVSSEN GNYDYLMYAD
IDYDHPDVVN EMKKWGVWYA NEVGLDGYRL DAVKHIKFSF
LKDWVDNARA ATGKEMFTVG EYWQNDLGAL NNYLAKVNYN
QSLFDAPLHY NFYAASTGGG YYDMRNILNN TLVASNPTKA
VTLVENHDTQ PGQSLESTVQ PWFKPLAYAF ILTRSGGYPS
VFYGDMYGTK GTTTREIPAL KSKIEPLLKA RKDYAYGTQR
DYIDNPDVIG WTREGDSTKA KSGLATVITD GPGGSKRMYV
GTSNAGEIWY DLTGNRTDKI TIGSDGYATF PVNGGSVSVW
VQQ

Using SEQ ID NO: 2 as a starting point, a number of combinatorial CspAmy2 variants were previously made and tested, as described in WO/2014/164777. The best performing variants generally included a stabilizing mutation at an amino acid position corresponding to either E187 or S241, along with additional mutations that improved desirable properties.

In some embodiments, the present compositions and methods involve variant CspAmy2 α-amylases having a mutation at one or more of the positions corresponding to E187, S241, N126, F153, T180, E187, and 1203, optionally in combination with mutations at amino acid residue corresponding to R377, S362 and/or Y303.

In some embodiments, the particular mutations included in the variants are E187P, S241Q, N126Y, F153W, T180H, T180D, E187P, I203Y, Y303A, R377Y and S362A, R377Y, S362A and/or Y303A. In some embodiments, the variant α-amylase further includes one or more previously described mutations at an amino acid residue corresponding to G476, G477, E132, Q167, A277, R458, T459, and/or D460. Particular combinatorial variants include but are not limited to CspAmy2-C16E having a deletion of residues R178 and G179 and the substitutions N126Y, F153W, T180H, E187P and 1203Y, C16E-AY, further having the substitutions S362A and R377Y and C16E-AY-Y303A, further having the substitution Y303A. These combinatorial variant are described in WO 2017/100720.

The reader will appreciate that where an α-amylase naturally has a mutation listed above (i.e., where the wild-type α-amylase already comprised a residue identified as a mutation), then that particular mutation does not apply to that α-amylase. However, other described mutations may work in combination with the naturally occurring residue at that position.

In some embodiments, the present α-amylase variants have the indicated combinations of mutations and a defined degree of amino acid sequence homology/identity to SEQ ID NO: 1 or SEQ ID NO: 2, for example, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% amino acid sequence homology/identity.

In some embodiments, the present α-amylase variants have the indicated combinations of mutations and are derived from a parental amylase having a defined degree of amino acid sequence homology/identity to SEQ ID NO: 1 or SEQ ID NO: 2, for example, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% amino acid sequence homology/identity.

Furthermore, the present α-amylase may include any number of conservative amino acid substitutions. Exemplary conservative amino acid substitutions are described in countless publications.

The present α-amylase may also be derived from any of the above-described amylase variants by substitution, deletion or addition of one or several amino acids in the amino acid sequence. for example less than 10, less than 9, less than 8, less than 7, less than 6, less than 5, less than 4, less than 3, or even less than 2 substitutions, deletions or additions. Such variants should have the same activity as α-amylase from which they were derived.

The present amylase may be “precursor,” “immature,” or “full-length,” in which case they include a signal sequence, or “mature,” in which case they lack a signal sequence. Mature forms of the polypeptides are generally the most useful. Unless otherwise noted, the amino acid residue numbering used herein refers to the mature forms of the respective amylase polypeptides. The present amylase polypeptides may also be truncated to remove the N or C-termini, so long as the resulting polypeptides retain amylase activity.

The present amylase may be a “chimeric.” “hybrid” or “domain swap” polypeptide, in that it includes at least a portion of a first α-amylase polypeptide, and at least a portion of a second α-amylase polypeptide. The present amylases may further include heterologous signal sequence, an epitope to allow tracking or purification, or the like.

In another aspect, the α-amylase is encoded by a nucleic acid having a specified amount of sequence identity to a polynucleotide encoding an α-amylase. An exemplary nucleic acid is provided as SEQ ID NO: 3, shown below (the underlined sequence encodes a LAT signal peptide).

ATGAAACAACAAAAACGGCTTTACGCCCGATTGCTGACGCTGTTATTTGCG
CTCATCTTCTTGCTGCCTCATTCTGCAGCTAGCGCAGCAGCGACAAACGGA
ACAATGATGCAGTATTTCGAGTGGTATGTACCTAACGACGGCCAGCAATGG
AACAGACTGAGAACAGATGCCCCTTACTTGTCATCTGTTGGTATTACAGCA
GTATGGACACCGCCGGCTTATAAGGGCACGTCTCAAGCAGATGTGGGGTAC
GGCCCGTACGATCTGTATGATTTAGGCGAGTTTAATCAAAAAGGTACAGTC
AGAACGAAGTATGGCACAAAAGGAGAACTTAAATCTGCTGTTAACACGCTG
CATTCAAATGGAATCCAAGTGTATGGTGATGTCGTGATGAATCATAAAGCA
GGTGCTGATTATACAGAAAACGTAACGGCGGTGGAGGTGAATCCGTCTAAT
AGAAATCAGGAAACGAGCGGCGAATATAATATTCAGGCATGGACAGGCTTC
AACTTTCCGGGCAGAGGAACAACGTATTCTAACTTCAAATGGCAGTGGTTC
CATTTTGATGGAACGGATTGGGACCAGAGCAGAAGCCTCTCTAGAATCTTC
AAATTCACGGGAAAGGCGTGGGACTGGGAGGTTTCTTCAGAAAACGGAAAT
TATGACTATCTGATGTACGCGGACATTGATTATGACCATCCGGATGTCGTG
AATGAAATGAAAAAGTGGGGCGTCTGGTATGCCAACGAAGTTGGGTTAGAT
GGATACAGACTTGACGCGGTCAAACATATTAAATTTAGCTTTCTCAAAGAC
TGGGTGGATAACGCAAGAGCAGCGACGGGAAAAGAAATGTTTACGGTTGGC
GAATATTGGCAAAATGATTTAGGGCCCTGAATAACTACCTGGCAAAGGTAA
ATTACAACCAATCTCTTTTTGATGCGCCGTTGCATTACAACTTTTACGCTG
CCTCAACAGGGGGTGGATATTACGATATGAGAAATATTCTTAATAACACGT
TAGTCGCAAGCAATCCGACAAAGGCTGTTACGTTAGTTGAGAATCATGACA
CACAGCCTGGACAATCACTGGAATCAACAGTCCAACCGTGTTTAAACCGTT
AGCCTACGCGTTTATTCTCACGAGAAGCGGAGGCTATCCTTCTGTATTTTA
TGGAGATATGTACGGTACAAAAGGAACGACAACAAGAGAGATCCCTGCTCT
TAAATCTAAAATCGAACCTTTGCTTAAGGCTAGAAAAGACTATGCTTATGG
AACACAGAGAGACTATATTGATAACCCGGATGTCATTGGCTGGACGAGAGA
AGGGGACTCAACGAAAGCCAAGAGCGGTCTGGCCACAGTGATTACAGATGG
GCCGGGCGGTTCAAAAAGAATGTATGTTGGCACGAGCAATGCGGGTGAAAT
CTGGTATGATTTGACAGGGAATAGAACAGATAAAATCACGATTGGAAGCGA
TGGCTATGCAACATTTCCTGTCAATGGGGGCTCAGTTTCAGTATGGGTGCA
GCAA

In some embodiments, the nucleic acid has at least 60%, at least 70%, at least 80%, at least 85%, at least 90%, at least 91%, at least 92%, at least 93%, at least 94%, at least 95%, at least 96%, at least 97%, at least 98%, or even at least 99% nucleic acid sequence identity to SEQ ID NO: 3.

In some embodiments, the nucleic acid hybridizes under stringent or very stringent conditions to a nucleic acid encoding (or complementary to a nucleic acid encoding) an α-amylase having at least 60%, at least 70%, at least 80%, at least 85%, at least 90%, at least 91%, at least 92%, at least 93%, at least 94%, at least 95%, at least 96%, at least 97%, at least 98% or even at least 99% nucleic acid sequence identity to SEQ ID NO: 3.

In some embodiments, the α-amylase for use in the compositions and methods has properties similar to CspAmy2 and its variants, which properties can be screened for under the conditions described, herein. As the unique properties of CspAmy2 in producing specialty syrups was heretofore unknown, the impetus to screen α-amylases for such properties was not recognized.

### 4 Elimination of the Need for Other Enzymes

Enzymes used in contemporary enzymatic processing conditions used to produce maltodextrin powder and specialty syrups are generally described, herein. The enzymes include maltogenic amylase (EC 3.2.1.133), 0-amylase (EC 3.2.1.2), pullulanase (EC 3.2.1.41) and glucoamylase (EC 3.2.1.3). The present compositions and methods reduce or obviate the need for one or more of these enzymes.

In some embodiments, the present compositions and methods reduce the need for any or all maltogenic enzymes by at least 50%, at least 60%, at least 70%, at least 80%, at least 85%, at least 90%, at least 95%, at least 97%, at least 98%, or even at least 99%, In some embodiments, the present compositions and methods entirely eliminate the need for any or all maltogenic enzymes in the producing maltodextrin powder and specialty syrups.

In some embodiments, the inclusion of trivial or inconsequential amounts of maltogenic enzymes for no significant benefit with respect to the present compositions and methods does not defeat the invention.

### 5 Simplified Process Conditions

Contemporary processing conditions used to produce maltodextrin powder and specialty syrups are generally described, herein. The present compositions and methods obviate the need for one of more process steps that are currently required for the enzymatic preparation of specialty syrups. For example, in various embodiments, the present compositions and methods obviate the need to reduce the pH of a liquefact to less than 4.50, less than 4.40, less than 4.30, or even less than 4.20, at 90° C., to inactivate a conventional liquifying α-amylase (which is an α-amylase that distinct from the present α-amylase), during the specialty syrup production process. In other embodiments, the present compositions and methods obviate the need to cool the liquefact to 55-65° C. for optimal performance of a glucoamylase or other maltogenic enzyme, following the use of the conventional α-amylase to perform liquefaction. In other embodiments, the present compositions and methods obviate the need to heat the saccharified liquifact to 85-90° C., e.g., 85° C., 86° C., 87° C., 88° C., 89° C., or 90° C., to inactivate the glucoamylase or other maltogenic enzyme. In other embodiments, the present compositions and methods obviate the need to cool the saccharified liquifact to 55-60° C. to concentrate the product to a desired level of DS.

Each of the steps that are obviated can be obviated separately or in combination, resulting in a minor or major improvement in existing specialty syrup production.

All references cited herein are herein incorporated by reference in their entirety for all purposes. In order to further illustrate the compositions and methods, and advantages thereof. the following specific examples are given with the understanding that they are illustrative rather than limiting.

### EXAMPLES

The methods disclosed herein are illustrated in the following examples. From the above discussion and these examples, one skilled in the art can ascertain the various embodiments of this disclosure, and without departing from the spirit and scope thereof, can make various changes and modifications of the methods and compositions disclosed herein to adapt it to various uses and conditions.

### Example 1

### Specialty Syrup Using Single Enzymatic Process

Starch slurry was prepared by weighing 60 g corn starch (Sigma Aldrich Catalogue #S4126) followed by addition of 132 g of water into 500 mL Erlenmeyer flask. Slurry pH was adjusted to 5.60±0.10 using 1 N HCl, followed by addition of SPEZYME® HT TG (a variant α-amylase from a Cytophaga sp. having the substitutions N126Y, F153W T80H, E187P, I203Y, Y303A, S362A, R377Y and the deletion of R178 and G179) in an amount of 0.45, 0.75, 1.00, 1.50, 2.00 and 2.50 kg/T of starch. The final slurry volumes were adjusted with water to 200 ml. Liquefaction was carried out at 92° C. with continuous mixing at 350 rpm for 4 hours in a water bath. The flasks were sampled at 4 hours for determination of the DP profile using HPLC and DE using Lane and Eynon's method for reducing power, in which the mixed Fehling's solution is titrated with sample using methylene blue as indicator. Fehling's solution was standardised using 1% w/v glucose solution. The result are shown in Table 1.

TABLE 1
Effect of SPEZYME ® HT TG dose on the
DE and DP profile of specialty syrup

| Dose | | | | | |
|---|---|---|---|---|---|
| (kg/T) | % DP1 | % DP2 | % DP3 | % DP4+ | % DE |
| 0.45 | 6.82 | 9.66 | 19.05 | 64.47 | 32.75 |
| 0.75 | 8.11 | 11.03 | 19.54 | 61.32 | 35.21 |
| 1.00 | 8.53 | 11.70 | 19.81 | 59.96 | 35.92 |
| 1.50 | 8.96 | 12.64 | 20.37 | 58.03 | 36.67 |
| 2.00 | 9.39 | 13.52 | 20.83 | 56.26 | 39.12 |
| 2.50 | 10.12 | 14.69 | 21.05 | 54.14 | 40.01 |

The addition of SPEZYME® HT TG to the starch liquefaction resulted in specialty syrup with DE values ranging from 32-40%, The DP profile of the specialty syrup obtained by single enzymatic process using SPEZYME® HT TG is similar to the syrup produced using acid hydrolysis method with or without maltogenic enzymes.

### Example 2

### Effect of Time and Dose for Production of Specialty Syrup with Varying DP Profile

Starch slurry was prepared by weighing 60 g corn starch (Sigma Aldrich Catalogue #S4126) followed by addition of 132 g of water into 500 mL Erlenmeyer flask. Slurry pH was adjusted to 5.60±0.10 using 1 N HCl, followed by addition of SPEZYME® HT TG in an amount of 2.40 and 2.90 kg/T of starch. The final slurry volumes were adjusted with water to 200 ml. Liquefaction was carried out at 92° C. with continuous mixing at 350 rpm for 24 hours in a water bath. The flasks were sampled at 6, 8, 10 and 24 hours for determination of DP profile using HPLC and DE using Lane and Eynon's Method. The result are shown in Table 2.

TABLE 2
Effect of Liquefaction time and SPEZYME ® HT
TG dose on the DP profile of specialaity syrup

| Dose | Time | | | | | |
|---|---|---|---|---|---|---|
| (kg/T) | (h) | % DP1 | % DP2 | %DP3 | % DP4+ | % DE |
| 2.40 | 6 | 11.02 | 16.52 | 21.92 | 50.54 | 39.65 |
| | 8 | 11.82 | 17.98 | 22.43 | 47.76 | 41.89 |
| | 10 | 12.47 | 19.07 | 22.84 | 45.62 | 42.42 |
| | 24 | 15.31 | 23.56 | 23.86 | 37.27 | 43.60 |
| 2.90 | 6 | 11.42 | 17.35 | 22.45 | 48.77 | 42.12 |
| | 8 | 12.30 | 18.98 | 22.98 | 45.74 | 43.68 |
| | 10 | 12.99 | 20.17 | 23.38 | 43.46 | 44.09 |
| | 24 | 16.12 | 24.84 | 23.92 | 35.11 | 46.45 |

The extended starch liquefaction with SPEZYME® HT TG results in syrup with higher DE contributed by increased DP1 and DP2 content. Increase in dosage of enzymes further enhances the DP1 and DP2 content of the specialty syrup. Specialty syrup with desired DP profile can be obtained by modulating the SPEZYME HT TG dose and liquefaction time.

### Example 3

### Effect of High Solids on DP Profile Progress in Making Specialty Syrup

Starch slurry was prepared by weighing 80 g corn starch (Sigma Aldrich Catalogue #S4126) followed by addition of 132 g of water into 500 mL Erlenmeyer flask. Slurry pH was adjusted to 5.60±0.10 using 1N HCl, followed by addition of SPEZYME® HT TG in an amount of 1.00, 1.50, 2.00, 2.50 and 2.90 kg of starch. The final slurry volumes were adjusted with bath water to 200 ml. Liquefaction was carried out at 92° C. with continuous mixing at 350 rpm for 24 hours in a water bath. The flasks were sampled at 6, 8, 10 and 24 hours for determination of DP profile using HPLC. The result are shown in Table 4.

TABLE 4
Effect of liquefaction time and SPEZYME ® HT TG dose on the DP
profile of speciality syrup at 40% w/v starch (as it basis) loading

| Dose | | | | | |
|---|---|---|---|---|---|
| (kg/T) | Time (h) | % DP1 | % DP2 | % DP3 | % DP4+ |
| 1.00 | 6 | 8.60 | 12.21 | 20.76 | 58.43 |
| | 8 | 9.17 | 13.21 | 21.28 | 56.34 |
| | 10 | 9.59 | 14.11 | 21.69 | 54.61 |
| | 24 | 11.22 | 17.38 | 22.88 | 49.58 |
| 1.50 | 6 | 9.51 | 13.86 | 21.43 | 55.20 |
| | 8 | 10.23 | 15.18 | 22.00 | 52.60 |
| | 10 | 10.79 | 16.33 | 22.45 | 50.43 |
| | 24 | 12.90 | 20.20 | 23.90 | 43.00 |
| 2.00 | 6 | 10.52 | 15.47 | 21.89 | 52.12 |
| | 8 | 11.31 | 17.01 | 22.45 | 49.23 |
| | 10 | 12.01 | 18.33 | 22.92 | 46.75 |
| | 24 | 14.23 | 21.99 | 23.98 | 39.80 |
| 2.50 | 6 | 11.11 | 16.48 | 22.17 | 50.23 |
| | 8 | 12.02 | 18.15 | 22.72 | 47.11 |
| | 10 | 12.73 | 19.47 | 23.11 | 44.69 |
| | 24 | 14.23 | 21.99 | 23.98 | 39.80 |
| 2.90 | 6 | 11.59 | 17.53 | 22.74 | 48.14 |
| | 8 | 12.66 | 19.34 | 23.29 | 44.70 |
| | 10 | 13.48 | 20.77 | 23.78 | 41.96 |
| | 24 | 16.07 | 24.81 | 24.41 | 34.71 |

The results obtained in Example 2 using 30% w/v starch were generally replicated using the 40% w starch.

### Example 4

### Effect of Different α-Amylases in Making Specialty Syrup

Starch slurry was prepared by weighing 80 g corn starch (Sigma Aldrich Catalogue #S4126) followed by addition of 132 g of water into 500 mL Erlenmeyer flask. Slurry pH was adjusted to 5.60±0.10 using 1 N HCl, followed by addition of SPEZYME® HT TG, SPEZYME® ALPHA, SPEZYME® RSL, SPEZYME® FRED in amount of 1.00 kg/T of starch. The final slurry volumes were adjusted with water to 200 ml. Liquefaction was carried out at 92° C. with continuous mixing at 350 rpm for 24 hours in a water bath. The flasks were sampled at 6 and 24 hours for determination of DP profile using HPLC. The result are shown in Table 5.

TABLE 5
Comparative assessment of different α-amylases for
producing speciality syrup at 40% w/v dry starch loading

| | Dose | Time | | | | |
|---|---|---|---|---|---|---|
| Enzyme | (kg/T) | (h) | % DP1 | % DP2 | % DP3 | % DP4+ |
| SPEZYME ® | 1.00 | 6 | 8.60 | 12.21 | 20.76 | 58.43 |
| HT TG | | 24 | 11.22 | 17.38 | 22.88 | 49.58 |
| SPEZYME ® | 1.00 | 6 | 4.20 | 12.16 | 15.04 | 68.60 |
| ALPHA | | 24 | 7.88 | 15.63 | 16.85 | 59.64 |
| SPEZYME ® | 1.00 | 6 | 3.46 | 11.74 | 14.84 | 69.96 |
| RSL | | 24 | 6.57 | 14.89 | 16.59 | 61.96 |
| SPEZYME ® | 1.00 | 6 | 3.31 | 12.68 | 16.05 | 67.96 |
| FRED | | 24 | 8.03 | 18.12 | 18.08 | 55.80 |

Notably, SPEZYME® HT TG was the only enzyme tested that could yield a specialty syrup with a DP profile similar to the syrup produced using acid hydrolysis method with or without maltogenic enzymes.

### Example 6

### Effect of pH 4.50 on DP Profile Progress in Making Specialty Syrup

Starch slurry was prepared by weighing 80 g corn starch (Sigma Aldrich Catalogue #S4126) followed by addition of 132 g of water into 500 mL Erlenmeyer flask. Slurry pH was adjusted to 4.50±0.10 using 1 N HCl, followed by addition of SPEZYME® HT TG 2.00 kg/T of starch. The final slurry volumes were adjusted with water to 200 ml. Liquefaction was carried out at 92° C. with continuous mixing at 350 rpm for 24 hours in a water bath. The flasks were sampled at 6 and 24 hours for determination of DP profile using HPLC. The result are shown in Table 6.

TABLE 6
Effect of liquefaction pH on the DP profile & DE of
speciality syrup using SPEZYME ® HT TG

| Dose | Slurry | Time | | | | |
|---|---|---|---|---|---|---|
| (kg/T) | pH | (h) | % DP1 | % DP2 | % DP3 | % DP4+ |
| 2.00 | 5.50 | 6 | 10.52 | 15.47 | 21.89 | 52.12 |
| | | 24 | 14.23 | 21.99 | 23.98 | 39.80 |
| 2.00 | 4.50 | 6 | 9.96 | 14.05 | 21.16 | 54.83 |
| | | 24 | 11.51 | 16.91 | 22.46 | 49.12 |

With extended starch liquefaction time, lower liquefaction pH resulted in lower DP1, DP2 and DP3 content as compared to higher liquefaction pH. DP profiles with SPEZYME® HT TG at low pH (i.e., 4.50) was better than the DP profile obtained by SPEZYME® A SPEZYME® RSL, and SPEZYME® FRED at a higher pH (i.e., 5.50).

All publications, patents, and patent applications cited herein are hereby incorporated by reference in their entireties for all purposes and to the same extent as if each individual publication, patent, or patent application were specifically and individually indicated to be so incorporated by reference.

What is claimed is:

1. A method for producing a maltodextrin and/or a specialty syrup comprising contacting a starch substrate with an α-amylase (EC 3.2.1.1) capable of producing, in the substantial absence of a maltogenic enzyme selected from the group consisting of maltogenic amylase (EC 3.2.1.133), β-amylase (EC 3.2.1.2), pullulanase (EC 3.2.1.41), glucoamylase (EC 3.2.1.3) and combinations, thereof, a syrup comprising a DE profile equivalent to the DE profile produced by conventional, multi-enzyme, acid pretreatment conditions that includes a maltogenic enzyme, wherein the method substantially obviates at least one pH adjustment or temperature adjustment step in an otherwise identical process utilizing a different, conventional liquifying α-amylase.

2. The method of claim 1, performed in the absence of a maltogenic enzyme, with the exception of the α-amylase, which may have maltogentic amylase activity.

3. The method of claim 2, performed in the absence of any maltogenic enzyme, with the exception of the α-amylase, which may have maltogentic amylase activity.

4. The method of any of claims 1-3, wherein the process step is selected from the group consisting of reducing the pH of a liquefact to inactivate a different, conventional liquifying α-amylase, cooling the liquefact to promote optimal performance of a maltogenic enzyme, heating a saccharified liquifact to inactivate the maltogenic enzyme, and cooling the saccharified liquifact to concentrate the product.

5. The method of any of claims 1-4, wherein the α-amylase is from a Cytophaga sp.

6. The method of any of claims 1-5, wherein the α-amylase is the α-amylase from Cytophaga sp. having the amino acid sequence of SEQ ID NO: 1, or a variant, thereof.

7. The method of any of claims 1-5, wherein the conventional liquifying α-amylase is from Bacillus.
